(19) United States
(12) Patent Application Publication
(10) Pub. No.: US 2018/0185360 A1
(43) Pub. Date: Jul. 5, 2018

(54) CGRP RECEPTOR ANTAGONIST COMPOUNDS FOR TOPICAL TREATMENT OF SKIN DISORDERS

(71) Applicant: GALDERMA RESEARCH & DEVELOPMENT, BIOT (FR)

(72) Inventors: Christelle NONNE, Sophia Antipolis (FR); Michel RIVIER, Nice (FR); Patricia ROSSIO, Grasse (FR); Feriel HACINI-RACHINEL, Biot (FR)

(21) Appl. No.: 15/741,044

(22) PCT Filed: Jun. 28, 2016
(86) PCT No.: PCT/EP2016/065062

§ 371 Date: Dec. 29, 2017

(30) Foreign Application Priority Data
Jun. 29, 2015 (EP) 15306032.2

(51) Int. Cl. A61K 31/499; A61P 17/06; A61P 17/00; A61P 9/14

(52) U.S. Cl. CPC A61K 31/499; A61P 9/14; A61P 17/00; A61P 17/06

(57) ABSTRACT

The use of a CGRP receptor antagonist compound of formula (I) for topical application is described for treating and/or preventing skin inflammatory pathologies with a neurogenic component. More specifically, use of a CGRP receptor antagonist compound of formula (I) is described for topical application for treating and/or preventing rosacea, especially type I rosacea, atopic dermatitis, psoriasis and/or acne.

The present invention relates to the use of CGRP receptor antagonist compounds and the pharmaceutical compositions comprising such compounds in dermatological field.

### BACKGROUND OF THE INVENTION

CGRP (Calcitonin gene-related peptide) is broadly expressed in the central and peripheral nervous system and is involved in various biological functions. There are more and more evidence that CGRP play a key role as neuromodulator, but it is mostly known as a strong vasodilatator. CGRP activates a heterodimer receptor (CGRP-R) composed of a class B protein-G coupled receptor (CLR or calcitonin-like receptor)) associated with a transmembrane protein modifying the receptor activity (RAMP1 or receptor activity-modifying protein 1). Many studies have been done on CGRP role and CGRP receptor activity. For instance, studies have demonstrated that migraine attack was associated to the release of CGRP by meningeal nociceptors of the trigeminal ganglion and that CGRP could play a key role in migraine attack.

The increasing number of evidences that CGRP was linked to migraine attack resulted in a high interest for the development of compounds able to block the effects of this neuropeptide. Important efforts have been made to develop direct small antagonist compounds of CGRP with vasoconstriction activity for treating migraine attack. These compounds are described in the literature for their oral use, especially olcegepant, telcagepant and MK-3207. However their development was discontinued since severe hepatotoxicity was associated to these compounds by oral route.

Besides its role in migraine attack, CGRP release by face trigeminal nerves could be involved in inflammation disorder responsible especially of permanent erythema in type I rosacea. This hypothesis is done knowing that rosacea and migraine are sharing common characteristics such as a neurogenic component with vasodilation phenomenon, the activation of trigeminal nervous system and the release of neuro-inflammatory peptides (CGRP, P substance . . . ). CGRP would have its vasodilatator action by acting through smooth muscle cells and sub-cutaneous vessels.

The compound 2-[(8R)-8-(3,5-difluorophenyl)-10-oxo-6,9-diazaspiro[4.5]dec-9-yl]-N-[R2R)-2′-oxo-1,1′,2′,3-tetrahydrospiro[indene-2,3′-pyrrolo[2,3-b]pyridin]-5-yl]acetamide also named MK-3207 developed by Merck & Co, is one of the most potent, orally bioavailable CGRP receptor antagonist described to date with a significantly higher efficiency for treating acute migraine in comparison with other CGRP receptor antagonist compounds such as telcagepant. Because of various toxic effects observed in patients during the clinical development of MK-3207, especially hepatotoxicity, its development has been discontinued.

Inflammatory skin diseases relate to skin diseases associated with inflammatory component. There are different types of inflammatory skin disease, classified by their localization, their causes and their symptoms. These skin disorders are common but their diagnosis can be difficult. Indeed, the skin immune system have limited way to respond to internal and external stimuli and many diseases present an inflammatory profile with few distinctive characteristics.

Rosacea is a common chronic and progressive inflammatory dermatosis associated with vascular relaxation. Rosacea affects principally central part of the face and is characterized by facial flushes, facial erythema, papules, pustules, telangiectasia and sometimes ocular lesions and rhinophyma. Moreover these primary features are associated with a secondary neurogenic component, more specifically to a cutaneous hyperreactivity of face and neck skin, characterized by the apparition of skin redness, prurit, feelings of itching, burning, stinging, and rough, flaky skin sensations.

Rosacea is classified into four subtypes according to the degree of primary features, such as vasomotor flushing, persistent erythema, papules and pustules, telangiectasias:

Erythematotelangiectatic rosacea (ETR) is mainly characterized by vasomotor flushing and persistent central facial erythema (redness). Telangiectasias (visible blood vessels) are commonly observed but are not essential for the diagnosis of this subtype. Central facial edema, burning or stinging sensations and rough, flaky skin are also symptoms that have sometimes been reported. A history of flushing as the only symptom is commonly found in people with erythematotelangiectatic rosacea. Facial flushing is due to the sudden dilatation of the arterioles of the face (which then takes a red appearance) and may be triggered by emotional stress, hot drink, alcohol, spicy food or temperature changes.

Papulopustular rosacea (PPR) is characterized by persistent central facial erythema and transient inflammatory crops of papules and/or pustules in the center of the face. However, the papules and pustules can also occur in periorificial regions, i.e., around the mouth, nose and eyes. The papulopustular subtype resembles acne vulgaris but comedones (specific of acne) are absent in rosacea. Rosacea and acne may coexist in a same patient, in which case comedones may also be present alongside the papules and pustules suggestive of rosacea. People with papulopustular rosacea sometimes complain of a burning or stinging sensation. Moreover, PPR is also characterized by the presence of inflammatory infiltrates that accompany flares, along with a heightened immune response involving neutrophilic infiltration and increased gene expression of IL8. This subtype is often observed after or at the same time as ETR (including the presence of telangiectasias).

Phymatous rosacea is characterized by a thickening of the skin, irregular surface nodularities and swelling. Patients with this subtype sometimes exhibit prominent, enlarged follicles as well as telangiectasias in the affected areas. The nose is most commonly affected (“rhinophyma”) but phymatous rosacea can also involve other areas such as the chin, the forehead, the cheeks and the ears. This subtype essentially affects men and often occurs after or at the same time as ETR or PPR.

Ocular rosacea (or ophthalmic rosacea) exhibits symptoms restricted to the ocular area with blepharitis, conjunctivitis and keratitis. It is characterized by watery or bloodshot eyes (interpalpebral conjunctival hyperemia), foreign body sensation, burning or stinging, dry or itchy eyes, sensitivity to light, blurred vision, conjunctival telangiectasias or eyelid margin telangiectasias or erythema of the eyelid and periocular area. They can occur with or without rosacea. The onset may occur before, during or after the onset of skin lesions.

The pathogenesis of rosacea is complex and not yet completely understood. Its etiology is multifactorial. In addition to exogenous factors (including UV light, temperature changes, alcohol, hormonal or emotional factors), it may be due to a higher density of Demodex folliculorum mites in rosacea patients. Such factors activate neurovascular and/or immune responses, and consequently inflammatory cascades. Intermittent flares may contribute to the chronicity of rosacea as they are associated with prolonged vasodilation, perivascular inflammation, edema and exposure to cytokines and cellular infiltrates. Moreover, many people who get rosacea have family history of the disease, suggesting a possible role of genetic.

Typical treatment of rosacea include oral or topical administration of antibiotics such as tetracycline, erythromycin, clindamycin, but also metronidazole (an antibacterial agent), low dose of isotretinoin in severe forms or even anti-infectious agents such as azelaic acid. However these treatments do not allow treating and/or preventing efficiently all the symptoms associated with rosacea, especially, the neurogenic component such as the skin hyperreactivity and redness.

In addition to rosacea, atopic dermatitis, psoriasis and acne belong to the most frequent inflammatory skin diseases.

Dermatitis derives from Greek language with “derma” meaning skin and “itis” meaning inflammation. Thus, dermatitis corresponds to skin inflammation that is classified in several specific and distinct types of dermatitis according to the localization, causes and symptoms thereof. Non exhaustive examples of dermatitis are atopic dermatitis, contact dermatitis, herpetiformis dermatitis, acrodermatitis, exfoliative dermatitis, perioral dermatitis, seborrheic dermatitis, eczema, hand eczema. Atopic dermatitis is a condition of the epidermis which affects a large number of individuals genetically predisposed to atopy, including infants, children and pregnant women.

Atopic dermatitis is an increasingly common pruritic inflammatory skin disorder due to complex interactions between the genetic predispositions and environmental factors. Atopic dermatitis has a complex etiology that involves abnormal immunological and inflammatory pathways that include defective skin barrier, exposure to environmental agents and neuropsychological factors. The diagnosis of atopic dermatitis is based on clinical presentation of skin erythematous plaques, eruption, and/or lichenification, typically in flexural areas accompanied by intense pruritus and cutaneous hypersensitivity. Pathological examination reveals spongiosis, hyperkeratosis and parakeratosis in acute lesions and marked epidermal hyperplasia, acanthosis, and perivascular accumulation of lymphocytes and mast cells (mastocytes) in chronic lesions.

Although the exact pathophysiology of atopic dermatitis has not yet been clearly understood, it has been reported that atopic dermatitis has at least two major components corresponding to a damaged skin barrier and a deregulated immune response.

Several authors have proposed pharmaceutical or dermatological compositions in order to treat symptoms of atopic dermatitis such as dry skin, erythematous plaques, eruption, lichenification, intense pruritus or cutaneous hypersensitivity and, to a greater extent, to compensate for the epithelial barrier deficiency. For example, pyrrolidone carboxylic acid (Takaoka, JP2004168763) and citrulline or certain amino acids such as glycine, methionine and alanine (Harano et al., WO2005/077349) have been used as a moisturizing agent in emollient compositions for treating atopic dermatitis. Tezuka (JP08020525) have proposed shampoos containing a complex of sodium montmorillonite with a moisturizing agent, which itself can be urea, amino acids, proteins, proteins, pyrrolidone carboxylic acid or a silk protein hydrolysate. Natural or synthetic immune inhibitors, anti-histamine agents, and steroids have also been used in pharmaceutical compositions in order to treat dermatitis atopic by reducing IgE production with the aim to reduce the immune response. For example, cyclosporin A holds the limelight as immune inhibitors or calcineurin inhibitor and is marked by Novartis under the name Sandimmun®. However, the conventional treating agents for atopic dermatitis using steroid and anti-histamine agents can only temporarily relieve symptoms. In addition, when topical or oral steroids are administered for a long term, the skin of a patient wears thin and osteoporosis is induced. Tacrolimus, which is marked under the names Prograf®, Advagraf® and Protopic®, may also be cited for treating atopic dermatitis as a calcineurin inhibitor leader. However, this drug is suspected of carrying a cancer risk and FDA (Food Drug Administration) has even issued a health warning in March 2005.

Psoriasis is an inflammatory skin disease characterized by red, itchy and scaly skin patches covered with white scales on top with a varying severity. These patches are mostly localized on the knees, elbows, scalp, and on lower back but can also affect other parts of the body. In this pathology, skin cells are quickly multiplied and then accumulated to form psoriatic patches. Psoriasis is characterized by an abnormally excessive and rapid growth of the epidermal skin cells which are replaced every 3-5 days rather than between 28-30 days in normal skin. This abnormal production of skin cells is thought to be due to premature maturation of keratinocytes induced by an inflammatory cascade in the dermis involving dendritic cells, macrophages and T cells. These immune cells are moving from the dermis to the epidermis to secrete cytokines which will stimulate keratinocyte proliferation. It has also been suggested that nerves play an important role in the pathogenesis of this disorder since the denervation of skin has shown an improvement in the psoriatic phenotype (Ostrowski et al., J. Invest. Dermatol., 2011, 131(7), 1530-1538).

Psoriasis pathology is multifactorial, with genetic pre-dispositions triggered by many environmental factors such as, stress, tobacco, alcohol, interruption of corticosteroids etc.

The current available treatments permit mostly to control the symptoms with a limited efficacy and may be use only during short time periods. For instance we can refer to topical treatments with corticosteroids, or with vitamin D3 analogous, retinoids, fluocinonides etc. However these treatments are not really efficient and generate several side effects when used during a long time period such as skin irritations and a pathology aggravation after treatment interruption. This disease affects 2-4% of the population without gender distinction.

Acne is also one of the most common inflammatory skin diseases. It is characterized by areas of skin with seborrhea (scaly red skin) and different types of lesions. Currently, acne lesions may be divided into non-inflammatory lesions (microcomedones, open and closed comedones), inflammatory lesions (papules, pustules, nodules, cysts) and possibly residual lesions or scarring (atrophic, hypertrophic and keloid scars). Lesions are most likely to occur on the face, neck, chest, shoulders and back, where there is a higher concentration of pilosebaceous units.

There exist several forms of acne, the common factor of all being attack of the pilosebaceous follicles. For example, we can mention acne vulgaris, neonatal acne, infantile acne, iatrogenic acne, cosmetic acne or severe forms like acne conglobata or acne fulminans. Acne vulgaris, commonly referred as “acne” or polymorphic juvenile acne, is the most common form and starts at puberty. This type of acne can be divided into mild, moderate, moderately severe and severe acne.

Despite extensive research on acne pathogenesis, the exact sequence of events and their possible mechanisms leading to the development of a microcomedone and its transformation into an inflamed lesion has remained unclear. (Shaheen B and Gonzales M, J of the European Academy of Dermatology and Venereology 2012). Recent reports unveiled the role of IL-17, and TNF-α pathways in the physiopathology of acne. Importantly, it has been shown that IL-17/Th17 pathway is activated in acne lesions. In addition, the pro-inflammatory cytokine TNF-α was significantly up-regulated in lesional acne (Kelhala H, Plos One 2014). Finally, Kistowska et al reported that acne patients exhibit stronger Th17 and Th17/Th1 (IFN-γ and TNF-α) responses to Propionibacterium acnes, a Gram-positive commensal bacterium thought to be involved in the pathogenesis of acne vulgaris (Kistowska et al. J Invest Dermatol. 2014).

Conventional treatments for treating acne include topical retinoids which reduce comedone formation and inflammatory response, topical or systemic antibiotics (such as erythromycin, clindamycin and tetracyclines) which aim is to reduce bacteria population and inflammation, benzoyl peroxide (BPO) which is both anti-bacterial and midly comedolytic. Using multiple agents (combined therapy) has been recommended to target as many pathophysiologic factors of acne. Oral isotretinoin, which targets all the pathophysiological factors involved in acne, is used in most severe and refractory cases because of serious side effects (such as teratogenicity or depression). However, these available therapies are harsh on the skin and can cause dryness, irritation, or redness.

In view of all these various elements, there is a need to find efficient compounds and develop new compositions for treating and/or preventing inflammatory skin pathologies, such as rosacea, atopic dermatitis, psoriasis and/or acne, on a long-term period and without toxic effects.

### SUMMARY OF THE INVENTION

The invention provides a safe treatment to discontinue or decrease skin inflammation, especially linked to a neurogenic inflammation. The invention provides a long-term effect with limited toxic effects especially regarding hepatotoxicity.

Applicant surprisingly demonstrated that compounds of formula (I), well known for their high activity as CGRP antagonist compounds by oral route but also for their hepatic toxic effects, were highly effective as CGRP antagonists by topical application besides avoiding their well-known side effects. More specifically it has been demonstrated that the topical application of these CGRP antagonist compounds is effective for treating and/or preventing skin inflammatory pathologies especially linked to a neurogenic component such as rosacea, atopic dermatitis, psoriasis and/or acne.

CGRP receptor antagonist compounds have been described in EP 2029575, as orally bioavailable drugs useful for treating acute migraine.

The present invention relates to CGRP receptor antagonist compound of formula (I) for topical application. More specifically the present invention relates to CGRP receptor antagonist compound of formula (I) for treating and/or preventing inflammatory skin pathologies with a neurogenic component by topical application. On another aspect the present invention relates to CGRP receptor antagonist compound of formula (I) for topical application for treating and/or preventing rosacea, atopic dermatitis and/or psoriasis. The CGRP receptor antagonist compound useful for the present invention is a compound of formula (I). This invention is also directed to a pharmaceutical composition for topical application, comprising at least one CGRP receptor antagonist compound of formula (I) and a pharmaceutically acceptable vehicle for the treatment and/or the prevention of skin inflammatory pathologies with a neurogenic component and especially rosacea, atopic dermatitis and/or psoriasis.

Compound according to the present invention for use in topically preventing or treating a dermatological disorder, are compound of following formula (I):

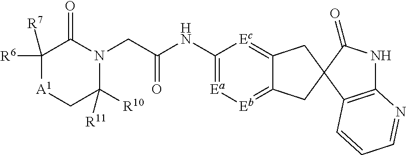

wherein:

Al is —CH2— or —N(R8)—;

Ea is selected from:

### (1) —C(R5a)═,

### (2) —N═, and

### (3) —(N+—O—)═;

Eb is selected from:

### (1) —C(R)═,

### (2) —N═, and

### (3) —(N+—O—)═;

Ec is selected from:

### (1) —C(R5c)═,

### (2) —N═, and

### (3) —(N+—O—)═;

R5a, R5b and R5c are each independently selected from:

(1) hydrogen,
(2) —C1-6alkyl, which is unsubstituted or substituted with 1-5 fluoro, and
(3) halo;

R6 and R7 are each:

(1) methyl which is unsubstituted or substituted with 1-3 fluoro; or
(2) ethyl, which is unsubstituted or substituted with 1-5 fluoro; or
(3) R6 and R7 and the carbon atom to which they are attached join to form a ring selected from cyclopropyl, cyclobutyl, cyclopentyl, cyclohexyl, cycloheptyl, tetrahydropyranyl, pyrrolidinyl, and piperidinyl, which ring is unsubstituted or substituted with 1-6 substituents each independently selected from:
(1) —C1-6alkyl, which is unsubstituted or substituted with 1-3 halo,
(2) phenyl, wherein the phenyl is optionally fused to the ring, and which phenyl is unsubstituted or substituted with 1-3 substituents each independently selected from: halo, —ORa, and —C1-4alkyl, which is unsubstituted or substituted with 1-3 fluoro, and
(3) halo;

R8 is independently selected from:

1) hydrogen,

### (2) —C(═O)Ra,

### (3) —CO2Ra,

### (4) —SO2Rd, and

(5) —C1-6alkyl, which is unsubstituted or substituted with 1-5 fluoro;

R10 is independently selected from:

(1) hydrogen,
(2—C1-6alkyl, which is unsubstituted or substituted with fluoro;

R11 is phenyl, which is unsubstituted or substituted with 1-5 substituents each independently selected from R12, R13, R14, R15a and R15b, R12, R13, R14, R15a and R15b are each independently selected from:

(1) —C1-6alkyl, which is unsubstituted or substituted with 1-5 substituents each independently selected from:

(a) halo,

(b) —ORa,

(c) —C3-6cycloalkyl,

(d) phenyl or heterocycle, wherein said heterocycle is selected from: pyridyl, pyrimidinyl, pyrazinyl, pyridazinyl, piperdinyl, piperazinyl, pyrrolidinyl, thienyl, morpholinyl, thiazolyl and oxazolyl, which phenyl or heterocycle is unsubstituted or substituted with 1-5 substituents each independently selected from:
(i) halo,
(ii) —C1-6alkyl, which is unsubstituted or substituted with 1-5 halo, and

(e) —CO2Ra,

(f) —C(═O)NRbRc,

(g) —S(O)vRd,

(h) —CN,

(i) —NRbRc,

(j) —N(Rb)C(═O)Ra,

(k) —N(Rb)SO2Rd,

(l) —CF3,

(m) —O—CO2Rd,

(n) —O—(C═O)—NRbRc,

(o) —NRb—(C═O)—NRbRc, and (p) —C(═O)Ra,

(2) —C1-6cycloalkyl, which is unsubstituted or substituted with 1-5 substituents each independently selected from:

(a) halo,

(b) —CN,

(c) —C1-6alkyl, which is unsubstituted or substituted with 1-5 halo,

(d) —ORa, and

(e) phenyl, which is unsubstituted or substituted with 1-5 substituents where the substituents are each independently selected from:
(i) —ORa,
(ii) halo,
(iii) —CN, and
(iv) —C1-6alkyl, which is unsubstituted or substituted with 1-5 halo,
   (3) phenyl or heterocycle, wherein said heterocycle is selected from: pyridyl, pyrimidinyl, pyrazinyl, pyridazinyl, piperdinyl, piperazinyl, pyrrolidinyl, thienyl, morpholinyl, thiazolyl and oxazolyl, which phenyl or heterocycle is unsubstituted or substituted with 1-5 substituents each independently selected from:

(a) halo,

(b) —ORa,

(c) —C3-6cycloalkyl,

(d) phenyl, which is unsubstituted or substituted with 1-5 substituents each independently selected from:
(i) halo,
(ii) —C1-6alkyl, which is unsubstituted or substituted with 1-6 halo, and
(iii) —ORa,

(e) —CO2Ra,

(f) —C(═O)NRbRc,

(g) —S(O)vRd,

(h) —CN,

(i) —NRbRc,

(j) —N(Rb)C(═O)Ra,

(k) —N(Rb)SO2Rd,

(l) —O—CO2Ra,

(m) —O—(C═O)—NRbRc,

(n) —NRb—(C═O)—NRbRc,

(o) —C(═O)Ra, and

(p) —C1-6alkyl, which is unsubstituted or substituted with 1-6 halo,

(4) halo,
(5) oxo,

### (6) —ORa,

### (7) —CN,

### (8) —CO2Ra,

### (9) —C(═O)Ra,

### (10) —NRbRc,

### (11) —S(O)vRd,

### (12) —C(═O)NRbRc,

### (13) —O—CO2Rd,

### (14) —N(Rb)CO2Rd,

### (15) —O—(C═O)—NRbRc,

### (16) —NRb—(C═O)—NRc,

### (17) —SO2NRbRc,

### (18) —N(Rb)SO2Rd,

or

R15a and R15b and the atom(s) to which they are attached join to form a ring selected from cyclopropyl, cyclobutyl, cyclopentyl, cyclohexyl, cycloheptyl, aziridinyl, azetidinyl, pyrrolidinyl, piperidinyl, piperazinyl, morpholinyl, thietanyl and tetrahydrothienyl, wherein the sulfur is optionally oxidized to the sulfone or sulfoxide, which ring is unsubstituted or substituted with 1-5 substituents each independently selected from:

(a) —C1-6alkyl, which is unsubstituted or substituted with 1-3 substituents each independently selected from:
(i) halo,
(ii) —ORa,
(iii) —C3-6cycloalkyl,
(iv) —CO2Ra,
(v) —NRbRc,
(vi) —S(O)vRd,
(vii) —C(═O)NRbRc, and
(viii) phenyl,

(b) phenyl or heterocycle, wherein said said heterocycle is selected from: pyridyl, pyrimidinyl, pyrazinyl, pyridazinyl, piperidinyl, piperazinyl, pyrrolidinyl, thienyl, morpholinyl, thiazolyl and oxazolyl, which phenyl or heterocycle is unsubstituted or substituted with 1-5 substituents each independently selected from:
(i) halo,
(ii) —C1-6alkyl, which is unsubstituted or substituted with 1-5 halo, and
(iii) —ORa,

(c) —ORa,

(d) halo,

(e) —CO2Ra,

(f) —C(═O)NRbRc,

(g) —S(O)vRd,

(h) —CN,

(i) —NRbRc,

(j) —N(Rb)C(═O)Ra,

(k) —N(Rb)SO2Rd,

(l) —O—CO2Rd,

(m) —O—(C═O)—NRbRc,

(n) —NRb—(C═O)—NRbRc, and

(o) —C(═O)Ra;

Ra is independently selected from:

(1) hydrogen,
(2) C1-6alkyl, which is unsubstituted or substituted with 1-7 substituents each independently selected from:

(a) halo,

(b) —O—C1-6alkyl, which is unsubstituted or substituted with 1-6 halo,

(c) hydroxyl,

(d) —CN, and

(e) phenyl or heterocycle wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
(i) halo,
(ii) —O—C1-6alkyl, which is unsubstituted or substituted with 1-6 halo,
(iii) —CN,
(iv) nitro, (v) hydroxyl, and
(vi) —C1-6alkyl, which is unsubstituted or substituted with 1-6 halo,
   (3) phenyl or heterocycle wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:

(a) halo,

(b) —CN,

(c) —O—C1-6alkyl, which is unsubstituted or substituted with 1-6 halo,

(d) nitro,

(e) hydroxyl, and

(f) —C1-6alkyl, which is unsubstituted or substituted with 1-6 halo, and

(4) —C3-6cycloalkyl, which is unsubstituted or substituted with 1-6 halo; Rb and Rc are independently selected from:

(1) hydrogen,

(2) C1-6alkyl, which is unsubstituted or substituted with 1-7 substituents each independently selected from:
(a) halo,
(b) —ORa,
(c) —CN,
(d) —CO2Ra,
(e) phenyl or heterocycle, wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
(i) halo,
(ii) —ORa,
(iii) —C1-6alkyl, which is unsubstituted or substituted with 1-6 halo, and
(iv) nitro,

(3) phenyl or heterocycle, wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:

(a) halo,

(b) —ORa,

(c) —C1-6alkyl, which is unsubstituted or substituted with 1-6 halo,

(d) —C3-6cycloalkyl, which is unsubstituted or substituted with 1-6 halo,

(e) —CN, and

(f) —CO2Ra,

(4) —C3-6cycloalkyl, which is unsubstituted or substituted with 1-6 halo; or Rb and Rc and the nitrogen to which they are attached join to form a 4-, 5-, or 6-membered ring optionally containing an additional heteroatom selected from N, O, and S, wherein the sulfur is optionally oxidized to the sulfone or sulfoxide, which ring is unsubstituted or substituted with 1-4 substituents each independently selected from:

(a) halo,

(b) —ORa, and

(c) —C1-6alkyl, which is unsubstituted or substituted with 1-6 halo, and

(d) phenyl;
Rd is independently selected from:
   (1) C1-6alkyl, which is unsubstituted or substituted with 1-4 substituents each independently selected from:

(a) halo,

(b) —ORa,

(c) —CO2Ra,

(d) —CN, and

(e) phenyl or heterocycle, wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
(i) halo,
(ii) —ORa,
(iii) —C1-6alkyl, which is unsubstituted or substituted with 1-6 halo, and
(iv) nitro,
   (2) phenyl or heterocycle, wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl tetrahydropyranyl and pyrazinyl, which phenyl or heterocycle is unsubstituted or substituted with 1-3 substituents each independently selected from:

(a) halo,

(b) —ORa,

(c) —C1-6alkyl, which is unsubstituted or substituted with 1-6 halo,

(d) —C3-6cycloalkyl, which is unsubstituted or substituted with 1-6 halo

(e) —CN, and

(f) —CO2Ra, and

(3) —C36cycloalkyl, which is unsubstituted or substituted with 1-6 halo; v is 0, 1, or 2; and pharmaceutically acceptable salts thereof and individual enantiomers and diastereomers thereof.

The compound of formula (I) are useful for topically treating and/or preventing dermatological disorder, especially skin inflammatory pathology with a neurogenic component.

According to the present invention, “dermatologic disorders” or “skin inflammatory pathologies with a neurogenic component” refers to skin inflammatory pathologies with a neurogenic component, selected from the group consisting of rosacea, especially type I erythematous rosacea, type II papulopustular rosacea, atopic dermatitis, hand chronic eczema, psoriasis (vulgaris, scalp, arthritic, pustular, guttate), facial erythema, pudic erythema, hives, urticaria (acute, chronic), any kind of pruritus (for instance senile pruritus, prurigo nodularis) and acne.

In a preferred embodiment, the skin inflammatory pathology with a neurogenic component is selected from the group consisting of rosacea, especially type I erythematous rosacea, type II papulopustular rosacea, atopic dermatitis, hand chronic eczema, psoriasis (vulgaris, scalp, arthritic, pustular, guttate), facial erythema, pudic erythema, hives (acute, senile pruritus, prurigo nodularis) and acne.

In a more preferred embodiment, the skin inflammatory pathology with a neurogenic component is selected from the group consisting of type I erythematous rosacea, atopic dermatitis, vulgaris and scalp psoriasis, acne, prurit senile and prurigo nodularis.

Even more preferably, the skin inflammatory pathology with a neurogenic component is type I erythematous rosacea, atopic dermatitis, psoriasis and/or acne.

According to a particular embodiment, the compound used according to the present invention is of formula (I) wherein:

Al is CH2 or —N(R8)—, wherein R8 is defined herein

Ea is —C(R5a)=, wherein R5a is defined herein, or Ea is —N═

Eb is —C(R5b)═, wherein R5b is defined herein or Eb is —N═

Ec is —C(R5c)═, wherein R5c is defined herein, or Ec is —N═

R5a, R5b and R5c are independently selected from hydrogen and halo

R6 and R7 are ethyl, unsubstituted or substituted with 1-5 fluoro or methyl unsubstituted or substituted with 1-3 fluoro, or selected from cyclopropyl, cyclobutyl, cyclopentyl, cyclohexyl, cycloheptyl, tetrahydropyranyl, pyrrolidinyl, and piperidinyl, which ring is unsubstituted or substituted with 1-6 substituents each independently selected from:
(1) —C1-6alkyl, unsubstituted or substituted with 1-3 halo,
(2) phenyl, optionally fused to the ring, unsubstituted or substituted with 1-3 substituents each independently selected from: halo, —ORa, and —C1-4alkyl, unsubstituted or substituted with 1-3 fluoro, and
(3) halo,
   wherein Ra is defined herein

R8 is selected from: hydrogen, and —C1-6alkyl, unsubstituted or substituted with 1-5 fluoro

R10 is hydrogen,

and pharmaceutically acceptable salts thereof and individual enantiomers and diastereomers thereof.

R11 is phenyl which is unsubstituted or substituted with 1-5 halo.

In a more specific embodiment, the compound according to the present invention is of formula (I) wherein:

Al is 1'N(R8)—, wherein R8 is defined herein

Ea is —C(R5a)═, wherein R5a is defined herein

Eb is —C(R5b)═, wherein R5b is defined herein

Ec is —C(R5c)═, wherein R5c is defined herein

R5a, R5b and R5c are hydrogen

R6 and R7 and the carbon atom to which they are attached join to form an unsubstituted cyclopentyl

R8 is hydrogen

R10 is hydrogen, and

R11 is a phenyl substituted with 1-5 fluor substituents.

Even more specifically, the preferred compound is 2-[(8R)-8-(3,5-difluorophenyl)-10-oxo-6,9-diazaspiro[4.5]dec-9-yl]-N-[(2R)-2′-oxo-1,1′,2′,3-tetrahydrospiro[indene-2,3′-pyrrolo[2,3-b]pyridin]-5-yl]acetamide. This compound is also commonly named MK3207 or CD10192/41 in the present description.

The present invention is also related to a pharmaceutical topical composition comprising at least a compound of formula (I), preferably compound MK3207, with a pharmaceutically acceptable vehicle suitable for topical application.

The pharmaceutical topical composition comprises from 0.001 to 10%, preferentially from 0.001 to 5%, more preferentially from 0.3 to 3%, and even more preferentially at 2 or 3% by weight of the compound of formula (I), especially MK 3207 relative to the total weight of the composition.

### LEGENDS TO THE FIGURES

FIG. 1: AUC Skin blood perfusion from 0 to 15 min (mean±sem).

After intravascular injection, αCGRP increased the basal blood perfusion in the skin by 242% (**p=0.0014). Topically administrated 90 minutes prior to the αCGRP induction, MK3207 inhibited this increase in a dose-dependent manner (**p=0.0017). Individually, MK3207 decreased the CGRP flush by 31% at 0.003% (NS, p=0.2259), 49% at 0.03% (NS, p=0.0567), 70% at 0.3% (*p=0.0112) and 80% at 3% (**p=0.0083).

FIG. 2: Graph plotting PD and PK mean for ear skin.

To improve the processing of data, the pharmacodynamic results are represented as a % inhibition of the perfusion compared to the values of the mouse group treated with Ethanol 100 (V002)/CGRP 20 μg/kg. The % inhibition can be negative in some cases: these values are therefore considered to be equal to 0 (absence of inhibition).

FIG. 3: PK/PD relationship for MK3207 with mean values per dose (PD). The black line represents the linear regression.

FIG. 4: Evaluation of the Effect of MK3207 in an Experimental Model of Atopic Dermatitis in Mouse
a) Effect of MK3207 on ear swelling on day 44
b) Effect of MK3207 on eosinophil peroxidase production on day 44
c) Effect of MK3207 on cytokine production on day 44
d) Effect of MK3207 on IFNγ production by T lymphocytes

FIG. 5: Evaluation of the Effect of MK3207 in an Experimental Imiquimod Induced Psoriasiform Mice Model
a) Effect of MK3207 on IL17-production within the γδ T cells
b) Effect of MK3207 on epidermis thickness

### DETAILED DESCRIPTION OF THE INVENTION

The applicant found that CGRP receptor antagonist compounds of family (I) are effective for treating and/or preventing inflammatory skin pathologies with a neurogenic component by topical application. More specifically, the applicant demonstrated that compounds of formula (I) are effective for treating and/or preventing skin inflammatory pathologies such as rosacea, especially type I rosacea, atopic dermatitis, psoriasis and/or acne by topical application. Even more specifically, the applicant demonstrated that the compound MK-3207 is effective for treating and/or preventing rosacea, especially type I rosacea, atopic dermatitis, psoriasis and/or acne by topical application.

Indeed, as demonstrated in the experimental section of the present description, the topical application of MK3207 allows decreasing the blood vessel dilation. The inventors more particularly observed a significant reduction of the blood skin perfusion with a dose and time dependent manner. Therefore the topical application of compound of formula (I) such as MK3207 is particularly useful for treating and/or preventing rosacea and especially type I rosacea, mainly characterized by permanent erythema and skin flushing induced by excessive blood vessel dilatation. Regarding another aspect, the topical application of MK3207 on an experimental model of atopic dermatitis mice demonstrated the capacity of MK3207 to decrease ear swelling as soon as day 27 with a similar effect until day 43. Moreover they observed that the production of inflammatory cytokines such as IL-4, IL-5, IL-13, TNFα and IL-17A were significantly reduced after topical application of MK3207. On another aspect, the MK3207 was also able to decrease the production of IFNγ by lymphocyte T cells. Thus, these results taken together demonstrate that the topical application of compound of formula (I) such as MK3207 is particularly useful for treating and/or preventing atopic dermatitis.

Imiquimod (IMQ) repeated topical application on mice skin has been shown to induce a skin inflammation resembling the human disease phenotype of psoriasis. Indeed, it induces inflamed scaly skin lesions resembling plaque type psoriasis. These lesions showed increased epidermal proliferation, abnormal differentiation, epidermal accumulation of neutrophils in microabcesses, neoangiogenesis, and infiltrates consisting of CD4(+) T cells, CD11c(+) dendritic cells, and plasmacytoid dendritic cells. IMQ induced epidermal expression of IL-23, IL-17A, and IL-17F, as well as an increase in splenic Th17 cells (J Immunol. 2009 May 1;182(9):5836-45). Applicant demonstrated in this model that the topical administration of MK3207 allows to significantly reduce the imiquimod induced epidermis increase and IL-17 production within the γδ T cells. These results also demonstrate that the topical application of compound of formula (I) such as MK3207 is also useful for treating and/or preventing psoriasis characterized by skin inflammation.

This topical application of CGRP receptor antagonist compounds of formula (I) for treating and/or preventing skin inflammatory disorders with a neurogenic component, especially rosacea, atopic dermatitis, psoriasis and/or acne allows to obtain a significant biological activity of CGRP receptor antagonist and a significant skin bioavailability with a significant hepatic metabolism instability allowing to avoid the toxic side effects observed by oral route.

Therefore the present application concerns CGRP receptor antagonist compounds of formula (I) for treating and/or preventing skin inflammatory pathologies with a neurogenic component, by topical application. More specifically the present invention concerns CGRP receptor antagonist compounds of formula (I) for treating and/or preventing rosacea, especially type I rosacea, atopic dermatitis and/or psoriasis by topical application. On a more specific aspect the present invention MK-3207 is the preferred compound. On another embodiment the present invention is also directed to a pharmaceutical composition for topical application, comprising at least one CGRP receptor antagonist compound of formula (I) and a pharmaceutically acceptable vehicle for the treatment and/or the prevention of skin inflammatory pathologies with a neurogenic component, and especially rosacea, atopic dermatitis and/or psoriasis. On another embodiment the present inventions concerns the topical use of CGRP receptor antagonists of formula (I). On a more specific embodiment the present invention concerns the topical use of CGRP receptor antagonists of formula (I) for treating and/or preventing skin inflammatory pathologies with a neurogenic component, especially rosacea, atopic dermatitis and/or psoriasis. On another aspect the present invention is directed to the use of a CGRP receptor antagonist of formula (I) for the manufacture of a medicament directed to treat and/or prevent skin inflammatory pathologies with a neurogenic component by topical application. Another aspect of the invention is directed to a process for treating and/or preventing skin inflammatory pathologies with a neurogenic component, comprising the topical administration of at least one compound of formula (I).

The present invention is further directed to a method for manufacturing a medicament comprising at least a compound of formula (I) with a pharmaceutical carrier or diluent for treating and/or preventing skin inflammatory pathologies with a neurogenic component such as rosacea and more especially type I rosacea but also atopic dermatitis and/or psoriasis.

The present invention is also related to a method for treating and or preventing skin inflammatory pathologies with a neurogenic component and more especially type I rosacea, but also atopic dermatitis and/or psoriasis by topical administration of a composition comprising the compound of formula (I) and more precisely MK3207.

CGRP receptor antagonist compounds of formula (I) according to the present invention, have been described in EP 2,029,575 B1.

According to the present invention, compounds for use in topically preventing and/or treating a dermatological disorder are compounds of formula (I):

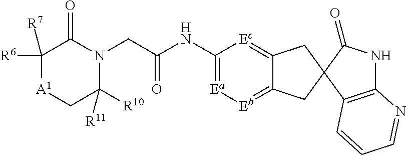

wherein:

Al is —CH2— or —N(R8)—;

Ea is selected from:

### (1) —C(R5a)═,

### (2) —N═, and

### (3) —(N+—O—)═;

Eb is selected from:

### (1) —C(R)═,

### (2) —N═, and

### (3) —(N+—O—)═;

Ec is selected from:

### (1) —C(R5c)═,

### (2) —N═, and

### (3) —(N+—O—)═;

R5a, R5band R5c are each independently selected from:

(1) hydrogen,
(2) —C1-6alkyl, which is unsubstituted or substituted with 1-5 fluoro, and
(3) halo;

R6 and R7 are each:

(1) methyl which is unsubstituted or substituted with 1-3 fluoro; or
(2) ethyl, which is unsubstituted or substituted with 1-5 fluoro; or

R6 and R7 and the carbon atom to which they are attached join to form a ring selected from cyclopropyl, cyclobutyl, cyclopentyl, cyclohexyl, cycloheptyl, tetrahydropyranyl, pyrrolidinyl, and piperidinyl, which ring is unsubstituted or substituted with 1-6 substituents each independently selected from:

(1) —C1-6alkyl, which is unsubstituted or substituted with 1-3 halo,
(2) phenyl, wherein the phenyl is optionally fused to the ring, and which phenyl is unsubstituted or substituted with 1-3 substituents each independently selected from: halo, —ORa, and —C1-4alkyl, which is unsubstituted or substituted with 1-3 fluoro, and
(3) halo;

R8 is independently selected from:

(1) hydrogen,

### (2) —C(═O)Ra,

### (3) —CO2Ra,

### (4) —SO2Rd, and

(5) —C1-6alkyl, which is unsubstituted or substituted with 1-5 fluoro;

R10 is independently selected from:

(1) hydrogen,
(2) —C1-6alkyl, which is unsubstituted or substituted with fluoro;

R11 is phenyl, which is unsubstituted or substituted with 1-5 substituents each independently selected from R12, R13, R14, R15a and R15b, R12, R13, R14, R15a and R15b are each independently selected from:

(1) —C1-6alkyl, which is unsubstituted or substituted with 1-5 substituents each independently selected from:

(a) halo,

(b) —ORa,

(c) —C3-6cycloalkyl,

(d) phenyl or heterocycle, wherein said heterocycle is selected from: pyridyl, pyrimidinyl, pyrazinyl, pyridazinyl, piperdinyl, piperazinyl, pyrrolidinyl, thienyl, morpholinyl, thiazolyl and oxazolyl, which phenyl or heterocycle is unsubstituted or substituted with 1-5 substituents each independently selected from:
(i) halo,
(ii) —C1-6alkyl, which is unsubstituted or substituted with 1-5 halo, and
(iii) —ORa,

(e) —CO2Ra,

(f) —C(═O)NRbRc,

(g) —S(O)vRd,

(h) —CN,

(i) —NRbRc,

(j) —N(Rb)C(═O)Ra,

(k) —N(Rb)SO2Rd,

(l) —CF3,

(m) —O—CO2Rd,

(n) —O—(C═O)—NRbRc, (o) —NRb—(C═O)—NRbRc, and

(p) —C(═O)Ra,

(2) —C1-6cycloalkyl, which is unsubstituted or substituted with 1-5 substituents each independently selected from:

(a) halo,

(b) —CN,

(c) —C1-6alkyl, which is unsubstituted or substituted with 1-5 halo,

(d) —ORa, and

(e) phenyl, which is unsubstituted or substituted with 1-5 substituents where the substituents are each independently selected from:
(i) —ORa,
(ii) halo,
(iii) —CN, and
(iv) —C1-6alkyl, which is unsubstituted or substituted with 1-5 halo,
   (3) phenyl or heterocycle, wherein said heterocycle is selected from: pyridyl, pyrimidinyl, pyrazinyl, pyridazinyl, piperdinyl, piperazinyl, pyrrolidinyl, thienyl, morpholinyl, thiazolyl and oxazolyl, which phenyl or heterocycle is unsubstituted or substituted with 1-5 substituents each independently selected from:

(a) halo,

(b) —ORa,

(c) —C3-6cycloalkyl,

(d) phenyl, which is unsubstituted or substituted with 1-5 substituents each independently selected from:
(i) halo,
(ii) —C1-6alkyl, which is unsubstituted or substituted with 1-6 halo, and
(iii) —ORa,

(e) —CO2Ra,

(f) —C(═O)NRbRc,

(g) —S(O)vRd,

(h) —CN,

(i) —NRbRc,

(j) —N(Rb)C(═O)Ra,

(k) —N(Rb)SO2Rd,

(l) —O—CO2Ra,

(m) —O—(C═O)—NRbRc,

(n) —NRb—(C═O)—NRbRc,

(o) —C(═O)Ra, and

(p) —C1-6alkyl, which is unsubstituted or substituted with 1-6 halo,

(4) halo,
(5) oxo,

### (6) —ORa,

### (7) —CN,

### (8) —CO2Ra,

### (9) —C(═O)Ra,

### (10) —NRbRc,

### (11) —S(O)vRd,

### (12) —C(═O)NRbRc,

### (13) —O—CO2Rd,

### (14) —N(Rb)CO2Rd,

### (15) —O—(C═O)—NRbRc,

### (16) —NRb—(C═O)—NRc,

### (17) —SO2NRbRc,

### (18) —N(Rb)SO2Rd,

or

R15a and R15b and the atom(s) to which they are attached join to form a ring selected from cyclopropyl, cyclobutyl, cyclopentyl, cyclohexyl, cycloheptyl, aziridinyl, azetidinyl, pyrrolidinyl, piperidinyl, piperazinyl, morpholinyl, thietanyl and tetrahydrothienyl, wherein the sulfur is optionally oxidized to the sulfone or sulfoxide, which ring is unsubstituted or substituted with 1-5 substituents each independently selected from:

(a) —C1-6alkyl, which is unsubstituted or substituted with 1-3 substituents each independently selected from:
(i) halo,
(ii) —ORa,
(iii) —C3-6cycloalkyl,
(iv) —CO2Ra,
(v) —NRbRc,
(vi) —S(O)vRd,
(vii) —C(═O)NRbRc, and
(viii) phenyl,

(b) phenyl or heterocycle, wherein said said heterocycle is selected from: pyridyl, pyrimidinyl, pyrazinyl, pyridazinyl, piperidinyl, piperazinyl, pyrrolidinyl, thienyl, morpholinyl, thiazolyl and oxazolyl, which phenyl or heterocycle is unsubstituted or substituted with 1-5 substituents each independently selected from:
(i) halo,
(ii) —C1-6alkyl, which is unsubstituted or substituted with 1-5 halo, and
(iii) —ORa,

(c) —ORa,

(d) halo,

(e) —CO2Ra,

(f) —C(═O)NRbRc,

(g) —S(P)vRd,

(h) —CN,

(i) —NRbRc,

(j) —N(Rb)C(═O)Ra,

(k) —N(Rb)SO2Rd,

(l) —O—CO2Rd,

(m) —O—(C═O)—NRbRc,

(n) —NRb—(C═O)—NRbRc, and

(o) —C(═O)Ra;

Ra is independently selected from:

(1) hydrogen,
(2) C1-6alkyl, which is unsubstituted or substituted with 1-7 substituents each independently selected from:

(a) halo,

(b) —O—C1-6alkyl, which is unsubstituted or substituted with 1-6 halo,

(c) hydroxyl,

(d) —CN, and

(e) phenyl or heterocycle wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
(i) halo,
(ii) —O—C1-6alkyl, which is unsubstituted or substituted with 1-6 halo,
(iii) —CN,
(iv) nitro, (v) hydroxyl, and
(vi) —C1-6alkyl, which is unsubstituted or substituted with 1-6 halo,
   (3) phenyl or heterocycle wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:

(a) halo,

(b) —CN,

(c) —O—C1-6alkyl, which is unsubstituted or substituted with 1-6 halo,

(d) nitro,

(e) hydroxyl, and
(f) —C1-6alkyl, which is unsubstituted or substituted with 1-6 halo, and
   (4) —C3-6cycloalkyl, which is unsubstituted or substituted with 1-6 halo; Rb and Rc are independently selected from:

(1) hydrogen,

(2) C1-6alkyl, which is unsubstituted or substituted with 1-7 substituents each independently selected from:
(a) halo,
(b) —ORa,
(c) —CN,
(d) —CO2Ra,
(e) phenyl or heterocycle, wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
(i) halo,
(ii) —ORa,
(iii) —C1-6alkyl, which is unsubstituted or substituted with 1-6 halo, and
(iv) nitro,

(3) phenyl or heterocycle, wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:

(a) halo,

(b) —ORa,

(c) —C1-6alkyl, which is unsubstituted or substituted with 1-6 halo,

(d) —C3-6cycloalkyl, which is unsubstituted or substituted with 1-6 halo,

(e) —CN, and

(f) —CO2Ra,

(4) —C3-6cycloalkyl, which is unsubstituted or substituted with 1-6 halo; or Rb and Rc and the nitrogen to which they are attached join to form a 4-, 5-, or 6-membered ring optionally containing an additional heteroatom selected from N, O, and S, wherein the sulfur is optionally oxidized to the sulfone or sulfoxide, which ring is unsubstituted or substituted with 1-4 substituents each independently selected from:

(a) halo,

(b) —ORa, and

(c) —C1-6alkyl, which is unsubstituted or substituted with 1-6 halo, and

(d) phenyl;

Rd is independently selected from:

(1) C1-6alkyl, which is unsubstituted or substituted with 1-4 substituents each independently selected from:

(a) halo,

(b) —ORa,

(c) —CO2Ra,

(d) —CN, and

(e) phenyl or heterocycle, wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
(i) halo,
(ii) —ORa,
(iii) —C1-6alkyl, which is unsubstituted or substituted with 1-6 halo, and
(iv) nitro,
   (2) phenyl or heterocycle, wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl tetrahydropyranyl and pyrazinyl, which phenyl or heterocycle is unsubstituted or substituted with 1-3 substituents each independently selected from:

(a) halo,

(b) —ORa,

(c) —C1-6alkyl, which is unsubstituted or substituted with 1-6 halo,

(d) —C3-6cycloalkyl, which is unsubstituted or substituted with 1-6 halo

(e) —CN, and

(f) —CO2Ra, and

(3) —C3-6cycloalkyl, which is unsubstituted or substituted with 1-6 halo; v is 0, 1, or 2; and pharmaceutically acceptable salts thereof and individual enantiomers and diastereomers thereof.

Halo or halogen as used herein are intended to include chloro, fluoro, bromo, and iodo.

As used herein, “alkyl” is intended to mean linear or branched structures having no carbon-to-carbon double or triple bonds. Thus C1-6alkyl is defined to identify the group as having 1, 2, 3, 4, 5 or 6 carbons in a linear or branched arrangement, such that C1-6alkyl specifically includes, but is not limited to, methyl, ethyl, n-propyl, iso-propyl, n-butyl, iso-butyl, tert-butyl, pentyl and hexyl. “Cycloalkyl” is an alkyl, part or all of which forms a ring of three or more atoms.

As used herein, “aryl” is intended to mean mono- or bi-cycle with 6 to 12 atoms of carbon of formula CₙH₍ₙ₋₂₎.

As used herein “heterocycloalkyl” is a cycloalkyl group as defined herein, comprising carbon and hydrogen atoms and at least one heteroatom, preferably, 1 to 4 heteroatoms selected from nitrogen, oxygen, and sulfur.

In an embodiment of the present invention Al is CH2 and other substituents are chosen among the various possibilities listed here above.

In an embodiment of the present invention Al is —N(R8)—, wherein R8 is defined herein and other substituents are chosen among the various possibilities listed here above.

In an embodiment of the present invention Al is —NH— and other substituents are chosen among the various possibilities.

In an embodiment of the present invention Ea is —C(R5a)═, wherein R5a is defined herein and other substituents are chosen among the various possibilities listed here above.

In an embodiment of the present invention Ea is —C(H)═ and other substituents are chosen among the various possibilities listed here above.

In an embodiment of the present invention Ea is —N═ and other substituents are chosen among the various possibilities listed here above.

In an embodiment of the present invention Eb is —C(R5b)═, wherein R5b is defined herein and other substituents are chosen among the various possibilities listed here above.

In an embodiment of the present invention Eb is —C(H)═ and other substituents are chosen among the various possibilities listed here above.

In an embodiment of the present invention Eb is —N═ and other substituents are chosen among the various possibilities listed here above.

In an embodiment of the present invention Ec is —C(R5c)═, wherein R5c is defined herein and other substituents are chosen among the various possibilities listed here above.

In an embodiment of the present invention Ec is —C(H)═ and other substituents are chosen among the various possibilities listed here above.

In an embodiment of the present invention Ec is —N═ and other substituents are chosen among the various possibilities listed here above.

In an embodiment of the present invention R5a, R5b and R5c are independently selected from hydrogen and halo, and other substituents are chosen among the various possibilities listed here above.

In an embodiment of the present invention R5a, R5b and R5c are hydrogen and other substituents are chosen among the various possibilities listed here above.

In an embodiment of the present invention R6 and R7 are ethyl, which are unsubstituted or substituted with 1-5 fluoro and other substituents are chosen among the various possibilities listed here above.

In an embodiment of the present invention R6 and R7 are methyl, which are unsubstituted or substituted with 1-3 fluoro and other substituents are chosen among the various possibilities listed here above.

In an embodiment of the present invention R6 and R7 and the carbon atom or atoms to which they are attached join to form a ring selected from cyclopropyl, cyclobutyl, cyclopentyl, cyclohexyl, cycloheptyl, tetrahydropyranyl, pyrrolidinyl, and piperidinyl, which ring is unsubstituted or substituted with 1-6 substituents each independently selected from:

(1) —C1-6alkyl, which is unsubstituted or substituted with 1-3 halo,

(2) phenyl, wherein the phenyl is optionally fused to the ring, and which phenyl is unsubstituted or substituted with 1-3 substituents each independently selected from: halo, —ORa, and —C1-4alkyl, which is unsubstituted or substituted with 1-3 fluoro, and

(3) halo,

wherein Ra is defined herein and other substituents are chosen among the various possibilities listed here above.

In an embodiment of the present invention R8 is selected from: hydrogen, and —C1-6 alkyl, which is unsubstituted or substituted with 1-5 fluoro and other substituents are chosen among the various possibilities listed here above.

In an embodiment of the present invention R8 is hydrogen and other substituents are chosen among the various possibilities listed here above.

In an embodiment of the present invention R8 is methyl and other substituents are chosen among the various possibilities listed here above.

In an embodiment of the present invention R10 is hydrogen and other substituents are chosen among the various possibilities listed here above.

It is to be understood that where one or more of the above recited structures or substructures recite multiple substituents having the same designation each such variable may be the same or different from each similarly designated variable.

In a specific embodiment, according to the present invention Al is —N(R8)—, wherein R8 is defined herein, Ea is —C(R5a)═, wherein R5a is defined herein, Eb is —C(R5b)═, wherein R5b is defined herein, Ec is —C(R5c)═, wherein R5c is defined herein, R5a, R5b and R5c are hydrogen, R6 and R7 and the carbon atom to which they are attached join to form a cyclopentyl, which ring is unsubstituted, R8 is hydrogen, R10 is hydrogen, and R11 is a phenyl substituted with 1-5 fluoro substituents.

According to the present invention, the preferred CGRP receptor antagonist compound for treating and/or preventing skin inflammatory pathologies with a neurogenic component, especially rosacea, is the compound with the following formula:

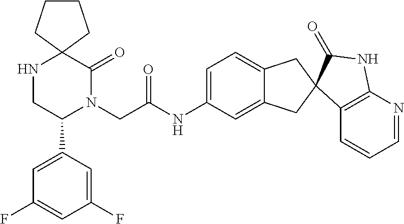 [SMILES: O=C(CN1C(=O)C2(CCCC2)NC[C@H]1c1cc(F)cc(F)c1)Nc1ccc2c(c1)C[C@@]1(C2)C(=O)Nc2ncccc21]

The chemical name of the preferred CGRP receptor antagonist compound according to the present invention is: 2-[(8R)-8-(3,5-difluorophenyl)-10-oxo-6,9-diazaspiro[4.5]dec-9-yl]-N-[2R)-2′-oxo-1,1′,2′,3-tetrahydrospiro[indene-2,3′-pyrrolo[2,3-b]pyridin]-5-yl]acetamide also commonly named MK-3207 or CD10192/41 and with the following CAS registry number: 957118-49-9.

The compounds of formula (I) may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention.

The compounds of formula (I) and their isomeric forms are useful according the present invention. The independent syntheses of these diastereomers or their chromatographic separations may be achieved as known in the art. Their absolute stereochemistry may be determined by the x-ray crystallography of crystalline products or crystalline intermediates which are derivatized, if necessary, with a reagent containing an asymmetric center of known absolute configuration. Racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The diasteromeric derivatives may then be converted to the pure enantiomers by cleavage of the added chiral residue. The racemic mixture of the compounds can also be separated directly by chromatographic methods utilizing chiral stationary phases, which methods are well known in the art. Alternatively, any enantiomer of a compound may be obtained by stereoselective synthesis using optically pure starting materials or reagents of known configuration by methods well known in the art.

All the chemical synthesis processes of these CGRP receptor antagonist compounds of formula (I) are such as described by Merck & Co in EP 2,029,575 B1 on page 15 to 65, more specifically on examples 1 to 21 and on scheme 1 to 26.

According to the present invention “pharmaceutically acceptable vehicle” refers to a vehicle appropriated for topical application, suitable for use in contact with the tissues of human beings and animals without excessive toxicity, irritation, allergic response, or other problem or complication, commensurate with a reasonable benefit/risk ratio. As used herein, “pharmaceutically acceptable salts” refer to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. In one aspect of the invention the salts are citric, hydrobromic, hydrochloric, maleic, phosphoric, sulfuric, fumaric, and tartaric acids. It will be understood that, as used herein, references to the compounds of Formula I are meant to also include the pharmaceutically acceptable salts.

According to the present invention “pharmaceutical composition” refers preferably to a dermatological composition which can be topically applied. More specifically “pharmaceutical composition” relates to a pharmaceutical composition comprising a CGRP receptor antagonist compound of formula (I) and a vehicle suitable for a topical application, for the treatment and/or the prevention of skin inflammatory pathology with a neurogenic component. MK-3207 is a preferred CGRP receptor antagonist compound of formula (I). Rosacea, especially type I rosacea, atopic dermatitis, psoriasis, and/or acne are the preferred skin inflammatory pathologies with a neurogenic component.

The choice of the concentration of CGRP receptor antagonist compounds of formula (I), preferably MK-3207, and the form of the pharmaceutical composition for the treatment and/or the prevention of a particular skin inflammatory pathology with a neurogenic component can be made depending on the type and severity of the pathology, location of the affected area, and form of the pharmaceutical composition. A person of ordinary skill in the art will be able to determine these different parameters. As an indication of useful range of concentration by weight, the compound of formula (I) and especially MK3207 can be used in a composition from 0.001 to 10%, preferentially from 0.001 to 5%, more preferentially from 0.3 to 3%, and even more preferentially at 2% or 3%, relative to the total weight of the composition.

The pharmaceutical composition is advantageously administered by topical application and, therefore, is in a form suitable for topical application to the skin. For example, it may be in the form of an optionally gelled, oily solution, an optionally two-phase dispersion of the lotion type, an emulsion obtained by dispersion of a fatty phase in an aqueous phase (O/W) or vice versa (W/O), or a triple emulsion (W/O/W or O/W/O) or a vesicular dispersion of ionic and/or non-ionic type. This topical composition may be in anhydrous form, in aqueous form or in the form of an emulsion. These compositions are prepared according to the usual methods. According to this invention, a composition in the form of an emulsion obtained by dispersion of a fatty phase in an aqueous phase (O/W) is preferably used.

This composition may be more or less fluid and may be in the form of salves, emulsions, creams, milks, ointments, impregnated pads, syndets, solutions, gels, sprays or aerosols, foams, suspensions, lotions or sticks. Preferably, the composition used in the present invention is in the form of an emulsion, of a cream, of a lotion type, of a gel, or of a solution, and more preferably in the form of an emulsion.

It is also considered that the pharmaceutical composition according to the present invention can be administered in combination with, or can comprise, an additional active ingredient or additive. The additional active ingredient is preferably selected from the group comprising antibiotics, antibacterial, antivirals, antiparasitics, antifungals, anesthetics, analgesics, antiallergic agents, retinoids, free-radical scavengers, anti-pruriginous, keratolytic agents, antiseborrheic, antihistaminic, sulfides, immunosuppressant products and antiproliferative agents, corticosteroids, intravenous immunoglobulin, anti-angiogenic, anti-inflammatory and/or a mixture thereof.

The additive is preferably selected from the group consisting of sequestering agents, chelating agents, antioxidants, sunscreens, preservatives, fillers, electrolytes, humectants, dyes, conventional acids, or bases, organic or inorganic, perfumes, essential oils, cosmetic active agents, moisturizers, vitamins, essential fatty acids, sphingolipids, self-tanning compounds, soothing and protective agents of the skin, penetrating agents, emulsifiers, gelling agents and a mixture thereof.

In one embodiment, the term “treatment” or “treating” refers to an improvement, the prophylaxis of a disease or disorder, or at least one symptom can be discerned therefrom. In another embodiment, “treatment” or “treating” means an improvement, prevention of at least one measurable physical parameter associated with the disease or disorder being treated, which is not necessarily discernible in the subject. In another further embodiment “treatment” or “treating” refers to inhibiting or slowing the progression of a disease or disorder, physically, e.g. stabilization of a discernible symptom, physiologically, for example, stabilization of a physical parameter, or both. In another embodiment, “treatment” or “treating” refers to delaying the onset of a disease or disorder. In some embodiments, compounds of interest are administered as a preventive measure. In this context, “prevention” or “preventing” refers to a reduction in the risk of acquiring a disease or disorder specified.

The present invention is directed to any mammal, particularly humans, male or female.

According to the present invention “skin inflammatory pathologies with a neurogenic component” refers to skin inflammatory pathologies with a neurogenic component chosen among rosacea such as type I erythematous rosacea, type II papulopustular rosacea, atopic dermatitis, hand chronic eczema, psoriasis (vulgaris, scalp, arthritic, pustular, guttate), facial erythema, pudic erythema, hives (acute, senile pruritus, prurigo nodularis) and acne.

More specifically the skin inflammatory pathologies with a neurogenic component are chosen among type I erythematous rosacea, atopic dermatitis, vulgaris and scalp psoriasis, acne, prurit senile and prurigo nodularis.

In a specific embodiment the skin inflammatory pathology with a neurogenic component according to the present invention is rosacea and especially type I erythematous rosacea, atopic dermatitis, and/or psoriasis.

In a more specific embodiment the skin inflammatory pathology with a neurogenic component according to the present invention is rosacea and especially type I erythematous rosacea. Type I erythematous rosacea according to the present invention is mainly characterized by persistent central facial erythema (redness), vasomotor flushing and telangiectasias (visible blood vessels). Central facial edema, burning or stinging sensations and rough, flaky skin are also symptoms that have sometimes been reported. Flushing is the only symptom commonly found in people with type I erythematous rosacea. Facial flushing is due to the sudden dilatation of the arterioles of the face (which then takes a red appearance) and may be triggered by emotional stress, hot drink, alcohol, spicy food or temperature changes.

In another more specific embodiment the skin inflammatory pathology with a neurogenic component according to the present invention is atopic dermatitis. In another more specific embodiment the skin inflammatory pathology with a neurogenic component according to the present invention is psoriasis.

Following examples illustrate the various aspects and advantages of the invention which are no way limiting in nature.

### EXAMPLES

### Example 1

### Evaluation of the Pharmacodynamic Activity of MK3207 (Herein Also Designated CD10192/41) Following Topical Treatment in a CGRP-Induced Mice Model of Vasodilatation

### Materials and Methods:

This study has been conducted on BALB/c mice, 5 mice per group have been used. Animals were topically treated with multiple doses of MK3207 (0.003% to 3%) in ethanol or with ethanol alone (20 μl, right ear), 90 minutes before the intravenous injection of αCGRP 20 μg/kg (2 hours before sampling).

Anaesthesia was performed 15 minutes before the intravenous injection of αCGRP or NaCl 0.9%. Body temperature was maintained at 35° C.-37° C. using an automated heating pad. Images were obtained from Laser Doppler perfusion imaging PIM3 (Perimed, France). For each mouse, the skin blood perfusion was evaluated every minute on the right ear. The laser beam of the PIM3 scanned a skin area of 2×2 cm. Prior to the treatment, 2 scans were performed.

The duration of the recording was 22 minutes in total.

The blood perfusion is mapped as colour coded images at intervals of 1 min. For each mouse ear, the area corresponding to the internal part of the ear is defined and adjusted for each image. From this selected area, we take the highest of the first two images as the background of blood perfusion (I ref). The background value is then subtracted from the next images of the kinetic.

The software calculates the erythematous surface (S) and the mean of the blood perfusion intensity (I) in this surface for the region of interest for all the images. The yield of the two parameters (I×S), the erythematous surface and the blood perfusion intensity ratio, was found to be the most relevant parameter. For each time of kinetic, the relative area of blood perfusion is calculated as follow: Relative area of perfusion=(I/I ref)×S

And the AUC is calculated from t=0 to t=15 min.

At the end of the scan, blood is collected from the abdominal artery on anesthetized animals. The animals are then euthanatized and the treated ear collected and weighted. Plasma and ear are snapped frozen.

### Results:

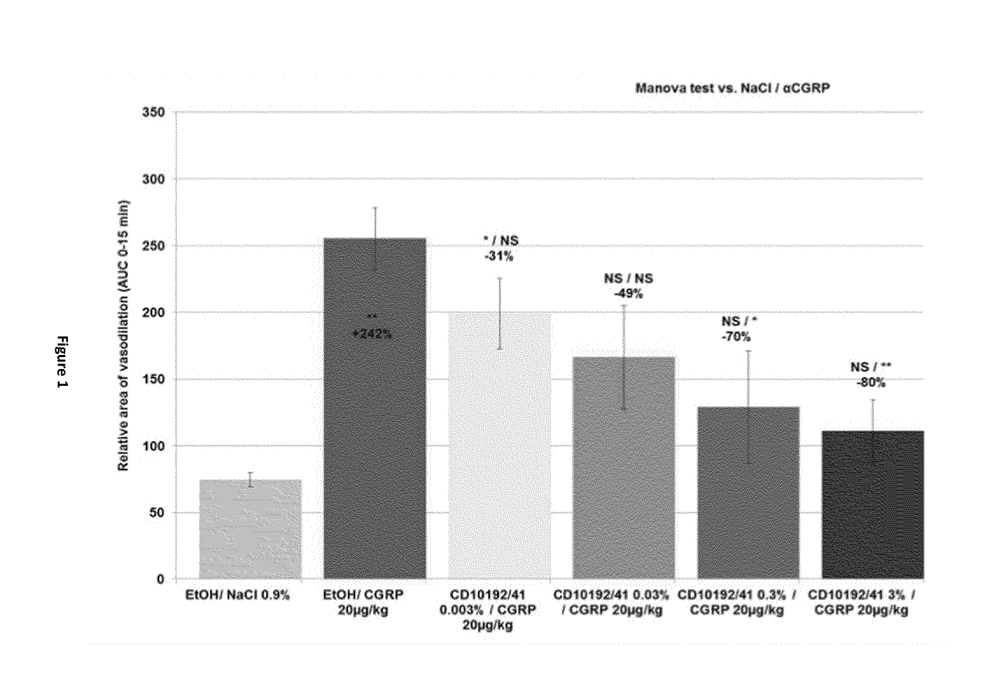

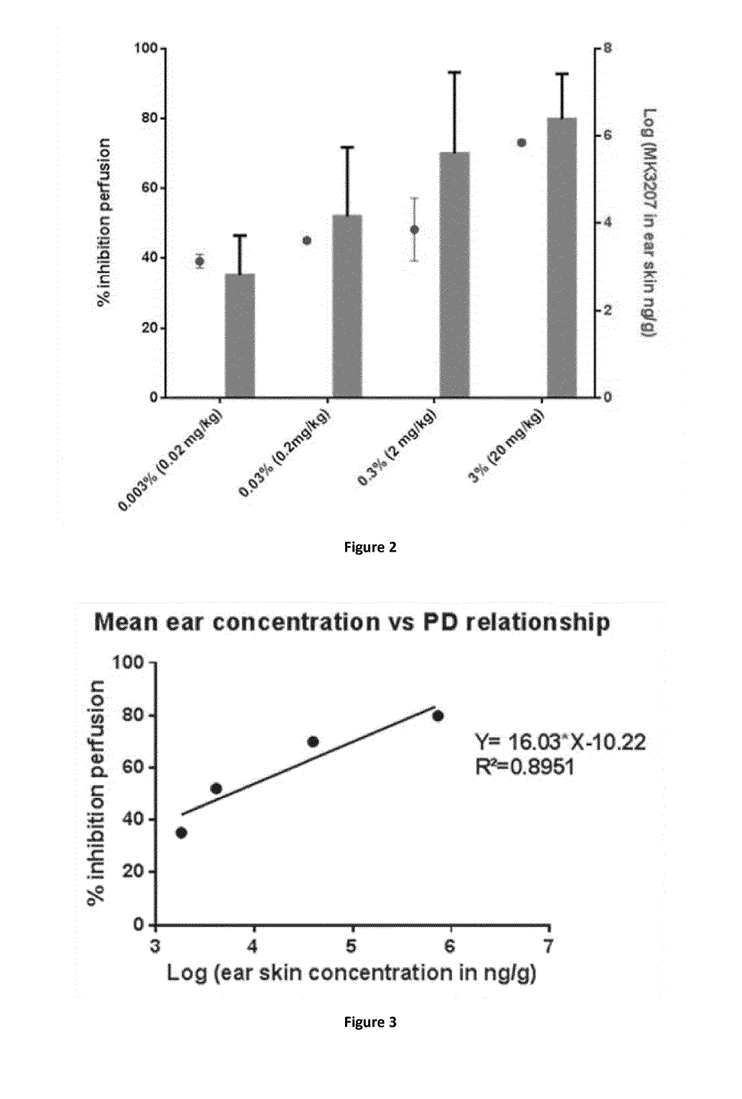

Results are presented in Table 1 below, and on FIGS. 1 to 3.

Two hours following a single topical application on the mouse ear, MK3207 was detected in skin samples at all doses tested. A dose-effect on skin concentrations was observed from 0.02 to 20 mg/kg (0.003% to 3%).

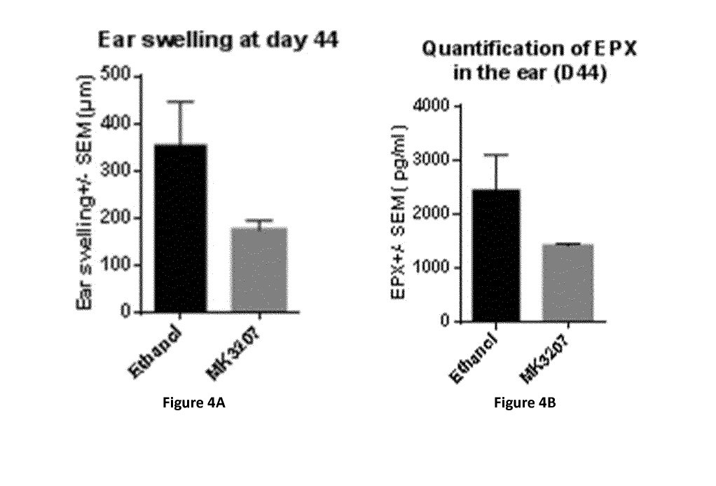

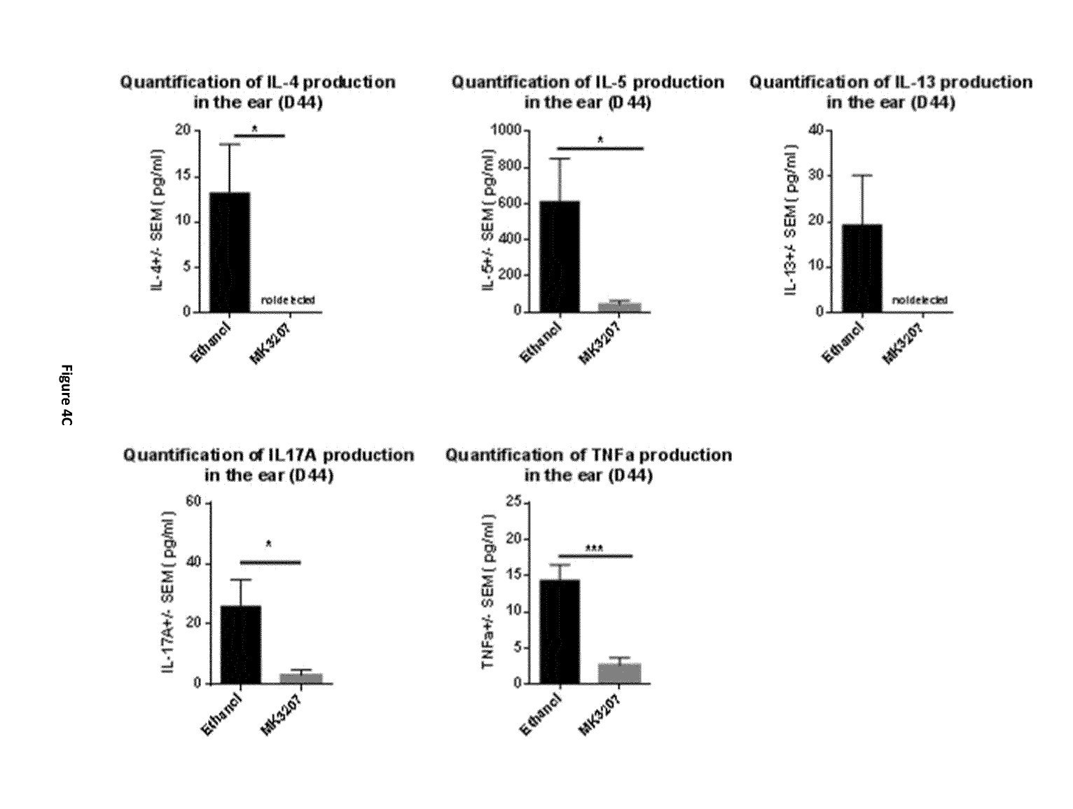

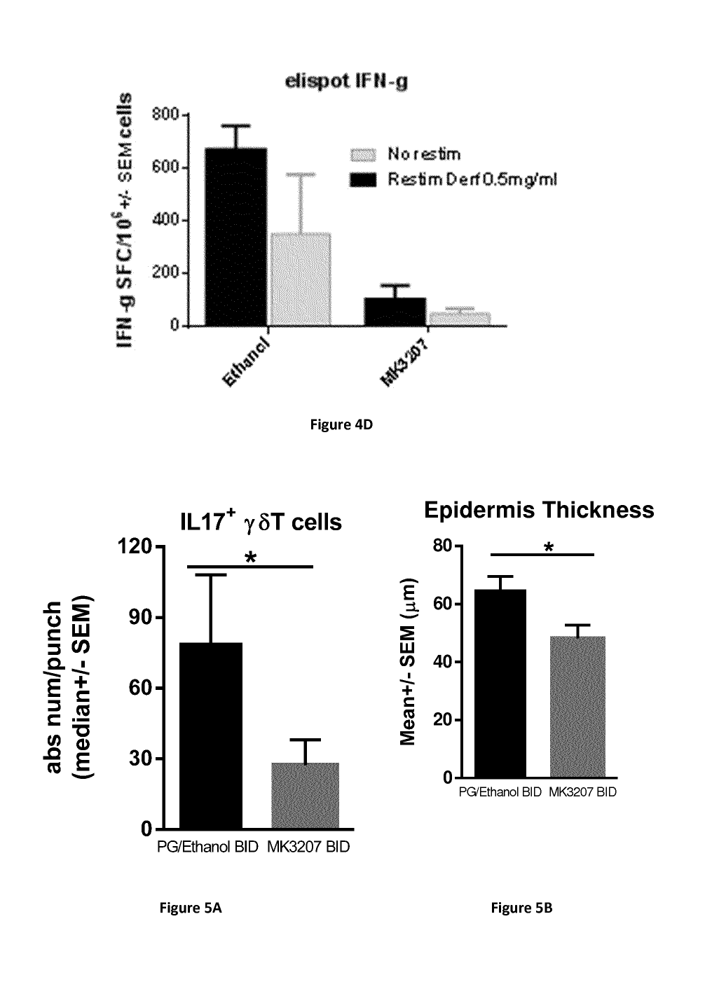

A linear trend is observed and also with the mean ear skin concentration values and the mean of the blood perfusion inhibition (FIG. 3, R²=0.8951).

To conclude the topical application of MK3207 at 0.003 to 3% at least 90 min before αCGRP induction, inhibits the blood skin perfusion increase. A dose-effect on skin concentration was observed from the doses of 0.2 to 20 mg/kg (0.003% to 3%).

These results demonstrate that topical MK3207 is useful for treating and/or preventing type I rosacea characterized by erythema and skin flushing induced by excessive blood vessel dilatation.

### Example 2

### Evaluation of the Anti-Inflammatory Effect of MK3207 (Herein Also Designated CD10192/41) in an Experimental Model of Atopic Dermatitis in Mouse

1. Material and methods:
1.1. Der F-Induced AD Mouse Model

The Dermatophagoides farinae (Der f)-induced AD mouse model has been shown to be suitable for the study of the atopic dermatitis pathophysiology and the evaluation of new therapies (J. Invest. Dermatol., 2009, 129, 31-40). Der f has been purchased from Greer Laboratories and a Der f solution has been prepared in 70% DMSO in MilliQ water. 375 μg of Der f has been applied during 7 weeks on the left ear of mice. Compounds to be tested were applied every day of the week except during the weekend, on the Der f treated ear from week 4 until day 44.
1.2. Compounds
   1.2.1. MK3207 (CD10192/41)

MK3207 (CD10192/41) has been provided by Galderma and 3% of MK3207 (CD10192/41) solutions in ethanol have been prepared.
1.2.2. Triamcinolone Acetonide

Triamcinolone acetonide has been chosen as positive control for the Der f model. 0.01% of triamcinolone acetonide solution in ethanol has been used.
1.2.3. Ethanol

Ethanol 100 has been chosen as a vehicle to solubilize the compounds.
1.3. Mice

Balb/c mice strain has been used for the study. These animals have been obtained from controlled breeding and were specific pathogen free. The animals have been provided by Janvier SAS, France.
1.4. Protocol

AD-like lesions have been induced by topical application of 375 μl of Der f solution on each side of the left ear for 7 weeks at day 1, day 8, day 15, day 22, day 29, day 36 and day 43. Mice have been treated from day 20 to day 43 five days by week with the different solutions administrated by the topical route.

Measurement of ear swelling has been monitored 24 hours after each allergen application. After day 19, ear swelling has been daily measured before compound application.

After mouse euthanasia, at Day 44 the left ear has been collected and has been washed in ethanol 70% and has been rinsed in PBS. Skin biopsies of the left ear, have then been collected for cytokines profile quantification with Luminex technique and for the quantification of the eosinophil peroxidase thanks to the ELISA method. Draining lymph nodes have been collected and cultivated a whole night restimulated or not with 0.5 mg/ml of Der f extract before quantifying the number of Der f specific lymphocytes T producing INFγ.

2. Results:

Der f/Ethanol group: In the negative control group, a significant increase in ear thickness, cytokine production and in INFγ production by T cell lymphocytes has been observed after Der f inducement.

Der f/Positive controls: As expected, triamcinolone acetonide inhibited the inflammatory response. The triamcinolone acetonide reduced ear edema, cytokine production and INFγ production by T cell lymphocytes.

Der f/MK3207 (CD10192/41): The topical application of MK3207 on an experimental model of atopic dermatitis mice demonstrated the capacity of MK3207 to decrease ear swelling as soon at day 44 (FIG. 4A). A reduction of the eosinophil peroxidase production (EPX) has also been observed at day 44 (FIG. 4B). Moreover, it has been observed that production of inflammatory cytokines such as IL-4, IL-5, IL-13, TNFα and IL17A were significantly reduced after topical application of MK3207 (FIG. 4C). Also, it has been observed that MK3207 was also able to decrease the production of IFNγ by lymphocyte T cells (FIG. 4D). Therefore the topical application of compound of formula (I) such as MK3207 is particularly useful for treating and/or preventing atopic dermatitis characterized by skin inflammation. These results also demonstrate that the topical application of compound of formula (I) such as MK3207 is also useful for treating and/or preventing, rosacea psoriasis, and acne, which are characterized by skin inflammation.

### Example 3

### Evaluation of the Effect of MK3207 (Herein Also Designated CD10192/41) in an Experimental Model of Psoriasiform Mice

1. Material and Methods
1.1 Imiquimod Induced Psoriasiform mice Model

Imiquimod psoriasiform model has been induced by daily topical application of Aldara (3.18 mg of imiquimod) for 3 or 7 days on the shaved back skin of Balb/c mice. The pharmacodynamic analysis were performed by using a transcriptional analysis at D4, a histological analysis at D4 and D8, a cells analysis at D8 and a Cytokines analysis at D8.
1.2. Compounds
   1.2.1. Imiquimod

Imiquimod is sold under the name Aldara™ 5% by 3M-Pharmaceutical. 63.5 mg of Aldara cream is applied on the back skin of the mice, representing 3.2 mg of active compound.
1.2.2. MK3207 (CD10192/41)

MK3207 compound is dissolved in PG/Ethanol 96 (30/70) and applied twice daily 2 h 00 before and 6 h 00 after Aldara treatment.
1.2.3. Vehicles

PG/Ethanol (30/70) is used as a vehicle.
1.2.4. Positive Control

CD0153F (Betamethasone valerate) at 0.01% (0.210 mM) in acetone by topical route is used as positive control.
1.3. Mice

Balb/c mice strain has been used for the study. These animals have been obtained from controlled breeding and were specific pathogen free. The animals have been provided by Janvier SAS, France.
1.4. Protocol

For the analysis at D4, the treatment was the following one from D1 to D3 and for the analysis at D8 the treatment was the following one from D1 to D7:

The negative control group was topically treated with 50 μl PG/Ethanol 96 2 hours before the topical application of 14 μL Aldara 5% and topically treated 50 μl with PG/Ethanol 96 6 hours after.

The positive control group was topically treated with 50 μl of CD0153F at 0.01% 2 hours before the topical application of 14 μL Aldara 5% on the Aldara application area.

The active group was topically treated with 50 μl of CD10192/41 at 2% 2hours before the topical application of 14 μL Aldara 5% and topically treated 50 μl of CD10192/41 at 2% with 6 hours after.

At day 4 skin biopsies were collected for quantifying gene expression of the inflammatory response via RT-PCR and for measuring epidermis thickness via histological stained slides (HE).

At day 8 blood samples and skin biopsies were collected for the characterization of the T-cells responses via the FACS technique and for measuring the cytokine production via a bead based assay.

2. Results

It has been observed at day 4 a reduction of 33% of the epidermis thickness with the compound CD10192/41 (FIG. 5B).

It has also been observed at day 8, a reduction of 55% of the number of IL17-production within the γδ T cells with the compound CD10192/41.

These results demonstrate that MK3207 compound is useful to treat and/or prevent psoriasis.

1. A method of treating a dermatological disorder, the method comprising topically administering an effective amount of a compound to an individual subject in need thereof, wherein the compound is a compound of formula I 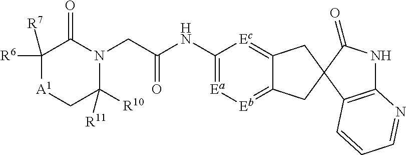
wherein:
Al is —CH2— or —N(R8)—;
Ea is selected from the group consisting of:
(1) —C(R5a)═,
(2) —N═, and
(3) —(N+—O—)═;
Eb is selected from the group consisting of:
(1) —C(R)═,
(2) —N═, and
(3) —(N+—O—)═;
Ec is selected from the group consisting of:
(1) —C(R5c)═,
(2) —N═, and
(3) —(N+—O—)═;
R5a, R5b and R5c are each independently selected from the group consisting of:
(1) hydrogen,
(2) —C1-6alkyl, which is unsubstituted or substituted with 1-5 fluoro, and
(3) halo;
R6 and R7 are each:
(1) methyl which is unsubstituted or substituted with 1-3 fluoro; or
(2) ethyl, which is unsubstituted or substituted with 1-5 fluoro; or
R6 and R7 and the carbon atom to which they are attached join to form a ring selected from the group consisting of cyclopropyl, cyclobutyl, cyclopentyl, cyclohexyl, cycloheptyl, tetrahydropyranyl, pyrrolidinyl, and piperidinyl, which ring is unsubstituted or substituted with 1-6 substituents each independently selected from the group consisting of:
(1) —C1-6alkyl, which is unsubstituted or substituted with 1-3 halo,
(2) phenyl, wherein the phenyl is optionally fused to the ring, and which phenyl is unsubstituted or substituted with 1-3 substituents each independently selected from the group consisting of: halo, —ORa, and —C1-4alkyl, which is unsubstituted or substituted with 1-3 fluoro, and
(3) halo;
R8 is independently selected from the group consisting of:
(1) hydrogen,
(2) —C(═O)Ra,
(3) —CO2Ra,
(4) —SO2Rd, and
(5) —C1-6alkyl, which is unsubstituted or substituted with 1-5 fluoro;
R10 is independently selected from the group consisting of:
(1) hydrogen, and
(2) —C1-6alkyl, which is unsubstituted or substituted with fluoro;
R11 is phenyl, which is unsubstituted or substituted with 1-5 substituents each independently selected from the group consisting of R12, R13, R14, R15a and R15b, R12, R13, R14, R15a and R15b are each independently selected from the group consisting of:
(1) —C1-6alkyl, which is unsubstituted or substituted with 1-5 substituents each independently selected from the group consisting of:
(a) halo,
(b) —ORa,
(c) —C3-6cycloalkyl, and
(d) phenyl or heterocycle, wherein said heterocycle is selected from the group consisting of: pyridyl, pyrimidinyl, pyrazinyl, pyridazinyl, piperdinyl, piperazinyl, pyrrolidinyl, thienyl, morpholinyl, thiazolyl and oxazolyl, which phenyl or heterocycle is unsubstituted or substituted with 1-5 substituents each independently selected from the group consisting of:
(i) halo,
(ii) —C1-6alkyl, which is unsubstituted or substituted with 1-5 halo, and
(iii) —ORa,
(e) —CO2Ra,
(f) —C(═O)NRbRc,
(g) —S(O)vRd,
(h) —CN,
(i) —NRbRc,
(j) —N(Rb)C(═O)Ra,
(k) —N(Rb)SO2Rd,
(l) —CF3,
(m) —O—CO2Rd,
(n) —O—(C═O—)—NRbRc,
(o) —NRb—(C═O)—NRbRc, and
(p) —C(═O)Ra,
(2) —C1-6cycloalkyl, which is unsubstituted or substituted with 1-5 substituents each independently selected from the group consisting of:
(a) halo,
(b) —CN,
(c) —C1-6alkyl, which is unsubstituted or substituted with 1-5 halo,
(d) —ORa, and
(e) phenyl, which is unsubstituted or substituted with 1-5 substituents where the substituents are each independently selected from the group consisting of:
(i) —ORa,
(ii) halo,
(iii) —CN, and
(iv) —C1-6alkyl, which is unsubstituted or substituted with 1-5 halo,
(3) phenyl or heterocycle, wherein said heterocycle is selected from the group consisting of: pyridyl, pyrimidinyl, pyrazinyl, pyridazinyl, piperdinyl, piperazinyl, pyrrolidinyl, thienyl, morpholinyl, thiazolyl and oxazolyl, which phenyl or heterocycle is unsubstituted or substituted with 1-5 substituents each independently selected from the group consisting of:
(a) halo,
(b) —ORa,
(c) —C3-6cycloalkyl, and
(d) phenyl, which is unsubstituted or substituted with 1-5 substituents each independently selected from the group consisting of:
(i) halo,
(ii) —C1-6alkyl, which is unsubstituted or substituted with 1-6 halo, and
(iii) —ORa,
(e) —CO2Ra,
(f) —C(═O)NRbRc,
(g) —S(O)vRd,
(h) —CN,
(i) —NRbRc,
(j) —N(Rb)C(═O)Ra,
(k) —N(Rb)SO2Rd,
(l) —O—CO2Ra,
(m) —O—(C═O)—NRbRc,
(n) —NRb—(C═O)—NRbRc,
(o) —C(═O)ORa, and
(p) —C1-6alkyl, which is unsubstituted or substituted with 1-6 halo,
(4) halo,
(5) oxo,
(6) —ORa,
(7) —CN,
(8) —CO2Ra,
(9) —C(═O)Ra,
(10) —NRbRc,
(11) —S(O)vRd,
(12) —C(═O)NRbRc,
(13) —O—CO2Rd,
(14) —N(Rb)CO2Rd,
(15) —O—(C═O)—NRbRc,
(16) —NRb—(C═O)—NRc, and
(17) —SO2NRbRc, (18) —N(Rb)SO2Rd, or
R15a and R15b and the atom(s) to which they are attached join to form a ring selected from the group consisting of cyclopropyl, cyclobutyl, cyclopentyl, cyclohexyl, cycloheptyl, aziridinyl, azetidinyl, pyrrolidinyl, piperidinyl, piperazinyl, morpholinyl, thietanyl and tetrahydrothienyl, wherein the sulfur is optionally oxidized to the sulfone or sulfoxide, which ring is unsubstituted or substituted with 1-5 substituents each independently selected from the group consisting of:
(a) —C1-6alkyl, which is unsubstituted or substituted with 1-3 substituents each independently selected from the group consisting of:
(i) halo,
(ii) —ORa,
(iii) —C3-6cycloalkyl,
(iv) —CO2Ra,
(v) —NRbRc,
(vi) —S(O)vRd,
(vii) —C(═O)NRbRc, and
(viii) phenyl,
(b) phenyl or heterocycle, wherein said said heterocycle is selected from the group consisting of: pyridyl, pyrimidinyl, pyrazinyl, pyridazinyl, piperidinyl, piperazinyl, pyrrolidinyl, thienyl, morpholinyl, thiazolyl and oxazolyl, which phenyl or heterocycle is unsubstituted or substituted with 1-5 substituents each independently selected from the group consisting of:
(i) halo,
(ii) —C1-6alkyl, which is unsubstituted or substituted with 1-5 halo, and
(iii) —ORa,
(c) —ORa,
(d) halo,
(e) —CO2Ra,
(f) —C(═O)NRbRc,
(g) —S(O)vRd,
(h) —CN,
(i) —NRbRc,
(j) —N(Rb)C(═O)Ra,
(k) —N(Rb)SO2Rd,
(I) —O—CO2Rd,
(m) —O—(C═O)—NRbRc,
(n) —NRb—(C═O)—NRbRc, and
(o) —C(═O)Ra;
Ra is independently selected from the group consisting of:
(1) hydrogen,
(2) C1-6alkyl, which is unsubstituted or substituted with 1-7 substituents each independently selected from the group consisting of:
(a) halo,
(b) —O—C1-6alkyl, which is unsubstituted or substituted with 1-6 halo,
(c) hydroxyl,
(d) —CN, and
(e) phenyl or heterocycle wherein said heterocycle is selected from the group consisting of pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from the group consisting of:
(i) halo,
(ii) —O—C1-6alkyl, which is unsubstituted or substituted with 1-6 halo,
(iii) —CN,
(iv) nitro, (v) hydroxyl, and
(vi) —C1-6alkyl, which is unsubstituted or substituted with 1-6 halo, and
(3) phenyl or heterocycle wherein said heterocycle is selected from the group consisting of pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from the group consisting of:
(a) halo,
(b) —CN,
(c) —O—6alkyl, which is unsubstituted or substituted with 1-6 halo,
(d) nitro,
(e) hydroxyl, and
(f) —C1-6alkyl, which is unsubstituted or substituted with 1-6 halo, and
(4) —C3-6cycloalkyl, which is unsubstituted or substituted with 1-6 halo; Rb and Rc are independently selected from the group consisting of:
(1) hydrogen,
(2) C1-6alkyl, which is unsubstituted or substituted with 1-7 substituents each independently selected from the group consisting of:
(a) halo,
(b) —ORa,
(c) —CN,
(d) —CO2Ra, and
(e) phenyl or heterocycle, wherein said heterocycle is selected from the group consisting of pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from the group consisting of:
(i) halo,
(ii) —ORa,
(iii) —C1-6alkyl, which is unsubstituted or substituted with 1-6 halo, and
(iv) nitro,
(3) phenyl or heterocycle, wherein said heterocycle is selected from the group consisting of pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from the group consisting of:
(a) halo,
(b) —ORa,
(c) —C1-6alkyl, which is unsubstituted or substituted with 1-6 halo,
(d) —C3-6cycloalkyl, which is unsubstituted or substituted with 1-6 halo,
(e) —CN, and
(f) —CO2Ra, and
(4) —C3-6cycloalkyl, which is unsubstituted or substituted with 1-6 halo; or Rb and Rc and the nitrogen to which they are attached join to form a 4-, 5-, or 6-membered ring optionally containing an additional heteroatom selected from the group consisting of N, O, and S, wherein the sulfur is optionally oxidized to the sulfone or sulfoxide, which ring is unsubstituted or substituted with 1-4 substituents each independently selected from the group consisting of:
(a) halo,
(b) —ORa, and
(c) —C1-6alkyl, which is unsubstituted or substituted with 1-6 halo, and
(d) phenyl;
Rd is independently selected from the group consisting of:
(1) C1-6alkyl, which is unsubstituted or substituted with 1-4 substituents each independently selected from the group consisting of:
(a) halo,
(b) —ORa,
(c) —CO2Ra,
(d) —CN, and
(e) phenyl or heterocycle, wherein said heterocycle is selected from the group consisting of pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from the group consisting of:
(i) halo,
(ii) —ORa,
(iii) —C1-6alkyl, which is unsubstituted or substituted with 1-6 halo, and
(iv) nitro,
(2) phenyl or heterocycle, wherein said heterocycle is selected from the group consisting of pyridyl, pyrimidinyl, thienyl, pyridazinyl, piperidinyl, azetidinyl, furanyl, piperazinyl, pyrrolidinyl, morpholinyl, tetrahydrofuranyl tetrahydropyranyl and pyrazinyl, which phenyl or heterocycle is unsubstituted or substituted with 1-3 substituents each independently selected from the group consisting of:
(a) halo,
(b) —ORa,
(c) —C1-6alkyl, which is unsubstituted or substituted with 1-6 halo,
(d) —C3-6cycloalkyl, which is unsubstituted or substituted with 1-6 halo
(e) —CN, and
(f) —CO2Ra, and
(3) —C3-6cycloalkyl, which is unsubstituted or substituted with 1-6 halo; v is 0, 1, or 2; and pharmaceutically acceptable salts thereof and individual enantiomers and diastereomers thereof.

2. The method according to claim 1, wherein the dermatological disorder is a skin inflammatory pathology with a neurogenic component.

3. The method according to claim 2, wherein the skin inflammatory pathology with a neurogenic component is selected from the group consisting of rosacea, atopic dermatitis, hand chronic eczema, psoriasis, facial erythema, pudic erythema, hives and acne.

4. The method according to claim wherein the skin inflammatory pathology with a neurogenic component is selected from the group consisting of type I erythematous rosacea, atopic dermatitis, vulgaris psoriasis, and scalp psoriasis, acne, prurit senile nodularis and prurigo nodularis.

5. The method according to claim 4, wherein the skin inflammatory pathology with a neurogenic component is type I erythematous rosacea, atopic dermatitis, psoriasis and/or acne.

6. The method according to claim 1, wherein:
Al is CH2 or —N(R8)—, wherein R8 is selected: hydrogen or C1-6alkyl, unsubstituted or substituted with 1-5 fluoro;
Ea is —C(R5a)═, or —N═;
Eb is —C(R5b)═, or —N═;
Ec is —C(R5c)═, or is —N═;
wherein R5a, R5b and R5c are independently hydrogen or halo;
R6 and R7 are ethyl, unsubstituted or substituted with 1-5 fluoro or methyl unsubstituted or substituted with 1-3 fluoro, or selected from the group consisting of cyclopropyl, cyclobutyl, cyclopentyl, cyclohexyl, cycloheptyl, tetrahydropyranyl, pyrrolidinyl, and piperidinyl, which ring is unsubstituted or substituted with 1-6 substituents each independently selected from the group consisting of:
(1) —C1-6alkyl, unsubstituted or substituted with 1-3 halo,
(2) phenyl, optionally fused to the ring, unsubstituted or substituted with 1-3 substituents each independently selected from the group consisting of: halo, —ORa, and —C1-4alkyl, unsubstituted or substituted with 1-3 fluoro, wherein Ra is as defined in claim 1; and
(3) halo,
R10 is hydrogen,
R11 is phenyl which is unsubstituted or substituted with 1-5 halo. and pharmaceutically acceptable salts thereof and individual enantiomers and diastereomers thereof.

7. The method according to claim 1, wherein:
Al is —N(R8)—, wherein R8 is hydrogen,
Ea is —C(R5a)═,
Eb is —C(R5b)═,
Ec is —C(R5c)═,
wherein R5a, R5b and R5c are hydrogen,
R6 and R7 and the carbon atom to which they are attached join to form an unsubstituted cyclopentyl,
R10 is hydrogen, and
R11 is a phenyl substituted with 1-5 fluor substituents.

8. The method according to claim 1, wherein the compound is 2-[(8R)-8-(3,5-difluorophenyl)-10-oxo-6,9-diazaspiro[4.5]dec-9-yl]-N-[(R2R)-2′-oxo-1,1′,2′,3-tetrahydrospiro[indene-2,3′-pyrrolo[2,3-b]pyridin]-5-yl]acetamide.

9. A topical pharmaceutical composition comprising at least a compound of formula (I) as defined in claim 1, and a pharmaceutically acceptable vehicle suitable.

10. The composition according to claim 9 wherein the compound is at a concentration ranging from about 0.001% to about 10%, by weight, relative to the total weight of the composition.

11. The composition according to claim 9 comprising about 3% of the compound of formula (I) by weight, relative to the total weight of the composition.

12. The composition according to claim 9 comprising about 2% of the compound of formula (I) by weight, relative to the total weight of the composition.

13. The method according to claim 3, wherein the rosacea is type I erythematous rosacea or type II papulopustular rosacea.

14. The method according to claim 3, wherein the psoriasis is vulgaris, scalp, arthritic, pustular, or guttate psoriasis.

15. The method according to claim 3, wherein the hives are acute, senile pruritus, or prurigo nodularis hives.

16. The composition according to claim 10, wherein the concentration of the compound is from about 0.001% to about 5%, by weight.

17. The composition according to claim 10, wherein the concentration of the compound is from about 0.3% to about 3%, by weight.
